(19) United States
(12) Patent Application Publication
(10) Pub. No.: US 2021/0030989 A1
(43) Pub. Date: Feb. 4, 2021

(54) RESPIRATORY MASK ASSEMBLY WITH A DYNAMIC CUFF

(71) Applicants: Kalyanaraman ANANTHANARAYANAN, TAMIL NADU (IN); KOTAK Nirav, Maharashtra (IN); PATYAL ASHISH, New Delhi (IN)

(72) Inventors: Kalyanaraman ANANTHANARAYANAN, TAMIL NADU (IN); KOTAK Nirav, Maharashtra (IN); PATYAL ASHISH, New Delhi (IN)

(21) Appl. No.: 16/968,217

(22) PCT Filed: Mar. 28, 2018
(86) PCT No.: PCT/IN2018/050177

§ 371 Date: Aug. 7, 2020

(30) Foreign Application Priority Data
Feb. 7, 2018 (IN) 201821004601

(51) Int. Cl. A61M 16/06; A61M 16/04; A61M 16/01

(52) U.S. Cl. CPC A61M 16/0605; A61M 16/0688; A61M 16/01; A61M 16/045

(57) ABSTRACT

This invention is a device to be used with a system for supplying anaesthetic or respiratory gases into the airway of a human or an animal. This device is a face mask, which includes a main body or ‘shell’ which is lined with one or more air filled cuffs of which at least one cuff is closely reflecting the airway pressures generated inside the shell during the various phases of the respiratory cycle and is inflated by the gases supplied directly from the anaesthesia or ventilator circuit through a specialised adaptor and a dedicated inflation port. Many other advantages of the invention will be apparent from reading the description which follows in conjunction with the accompanying drawings

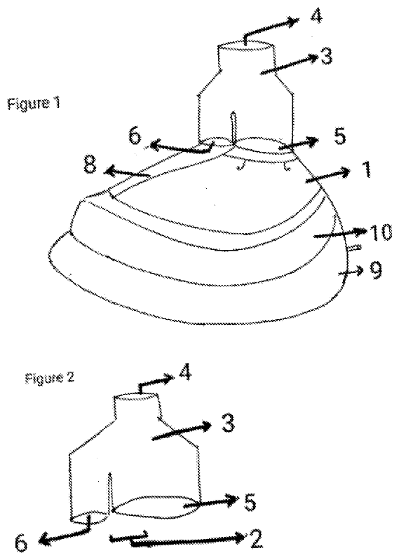

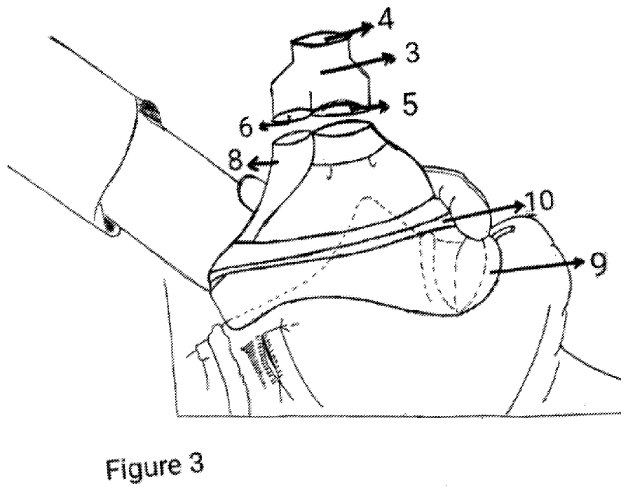

### THE FIELD OF THE INVENTION

The present invention relates to a facemask aids during positive pressure ventilation and its specialised sealing system.

This face mask “Mask” and its specialised adaptor, inflation system and the sealing cuffs are developed for providing ventilation and/or anaesthesia gases in the form of continuous or intermittent positive pressure ventilation (PPV), spontaneous respiration, jet ventilation or any such method of respiratory support, to a human or animal. Such a system can be used in patients under sedation/anaesthesia, or in patients on Non invasive ventilator (NIV) support in hospitalised patients or patients under home care, as required. The device can also be used in patients of Obstructive Sleep Apnea (OSA).

The term ‘mask’ hereby intended to include all respiratory devices which provide which provide a seal enabling positive pressure ventilation of a human or animal when applied on the face covering either a portion of or whole of the nose or mouth or both.

### BACKGROUND OF THE INVENTION

Normally, the work of breathing is done by the respiratory muscles, which suck in the air from the atmosphere into the lungs via the respiratory tract. This process is termed as “spontaneous respiration”. Respiration is divided into two phases, inspiration: which lasts roughly one third of a respiratory cycle, and expiration: which lasts the remaining two thirds of the respiratory cycle. Inspiration is an active process executed by the respiratory muscles trying to overcome the resistance offered by the patient's respiratory tract. Expiration is mostly a passive process.

In medical terminology, the region from the nostrils till the vocal cords is called the “upper respiratory tract” and the region from the vocal cords till the alveolar sacs in the lungs are called “lower respiratory tract”

When the patients have any respiratory tract ailments or are under the influence of sedation/anaesthesia, the ability of the respiratory muscles to supply air into the alveoli is decreased. In these situations, air may need to be “pumped” into the lungs using an external source which is called “Positive Pressure Ventilation”. The commonest of types of ventilation are via endotracheal tubes placed in the trachea, which directly pump the air into the lower respiratory tract (“Invasive Ventilation”) and via anatomical face masks placed on the face which aid to directly pump air/gases into the upper respiratory tract (“Non Invasive Ventilation” “NIV”).

The pressure in the patient's airway during positive pressure ventilation, which is required to push in the air into the lungs is called the “airway pressure”. It is generally highest during the inspiratory phase of the PPV and during the expiratory phase, it almost approaches atmospheric pressure.

Since Positive Pressure Ventilation in general, and Non Invasive Ventilation in particular, involves pushing in the air/gases into the lungs against the resistance offered by the patient's upper respiratory tract, the face mask should have an airtight seal when held/applied against the face, so that the air doesn't leak out while attempting to drive it into the patient's upper respiratory tract.

To achieve this, the face masks generally have a rigid body or “shell”, which has a proximal “Ventilator” end and a distal “Patient” end. The ventilator end has a standard 22 mm male/15 mm female port, which can be connected to the ventilator circuit. The other end is the distal “patient” end which has an inflatable cuff, which is filled with air, and when held tightly against the face over the nose and the mouth of the patient, will provide a reasonable air tight seal enabling air to be pumped into the patient's upper respiratory tract (Non invasive ventilation)

Several patients in hospital settings or in home care setting require positive pressure ventilation support. These patients can be the people who undergo intravenous sedation or anaesthesia for major/minor surgical procedures or admitted in hospital with respiratory ailments and the people requiring domiciliary ventilation support due to obstructive sleep apnea or other conditions.

Prior art these settings usually employ face mask with single inflatable cuff which mould to the contour of the face of the patient providing a reasonable seal against the face of the patient while held tightly. These face masks are either held against the face of the patient manually by the anaesthetist or by using a stretchable “Harness” belt in case of patients on Intensive Care on ventilator support.

Here the main problem is that the cuff will have to be pressed over the face of the patient at a pressure more than the Airway pressure to create an effective seal which is necessary to drive the anaesthetic/ventilator gases into the patient's airway, failing which the air will leak between the face mask and the face, as air always takes the path of least resistance.

The Airway pressure required to ventilate the lungs adequately in normal patients is under 20 cm of H20 (about 15 mm of Hg) but in certain situations such as patients with heavy mandible, obesity, Sleep apnea syndrome, patients with respiratory pathology, bronchospasm etc., the Airway pressure exceeds 40 cm of H20 or higher.

In such situations the mask unit will have to be held at much tighter (using more pressure on the cuff against the face) to maintain airtight seal/prevent air leak.

Even though the inspiratory phase which contributes to the peak airway pressure lasts typically one third of the duration of the respiratory cycle (the other two thirds being the exploratory phase), the masks available exert high “supra inspiratory pressures” throughout the respiratory cycle. This results in pressure of 40 cm of H20 or higher being exerted on the skin of the face of the patient for prolonged periods of time.

This 40 cm of H20 pressure exceeds the tissue perfusion pressure of the skin which is typically between 25 to 30 cm of H20. Prolonged compression of the face is not only very inconvenient for the patient but also can result in tissue injury, ischemia and necrosis.

Prior art in this field include face mask with inflatable cuffs and secured in place with hooks and harness belts. These harness belts are applied around the head of the patient and it is fixed with the hooks which are supplied on the mask, and this helps to keep the mask tightly pressed against the face of the patient. This is uncomfortable for the patient beyond a few minutes.

Even though the airway pressure is very low during expiratory phase of the respiratory cycle, which typically accounts for two-thirds of the respiratory cycle, or roughly 40 minutes during every hour of ventilation (or 16 hours of every day of Non invasive ventilation), the mask continuously exerts supra inspiratory airway pressures on the facial skin and tissues.

### OBJECTIVES AND ADVANTAGES OF THE INVENTION

Hence, it is an object of this invention is to provide a suitable face mask which can provide adequate seal throughout the respiratory cycle, but not to expose the facial skin to supra inspiratory pressures continuously.

It is another object of this invention make this device less inconvenient to the patient compared to the existing devices.

It is a further object of this device to prevent and tissue injury and necrosis due to prolonged, continuous compression of the facial skin and soft tissues compared to the earlier devices.

It is a further object of this invention to make this device compatible with all types of anaesthesia and ventilation systems which are used both in hospitalized patients and for patients under home care.

It is yet another object of this invention to make this device compatible with all monitoring equipment's, humidifiers, capnographs, anaesthesia gas sensors and temperature monitors.

In any face mask or similar ventilating device, certain amount of exhaled gases accumulates near the mouth of the patient which is driven into the patient's lungs during the next inspiration. This space is called “Dead Space” of the apparatus, Yet another object of this invention is to keep the ‘Apparatus Dead Space’ minimal so that rebreathing can be prevented.

### DETAILED DESCRIPTION OF THE INVENTION

Fig No 1 shows the device as viewed front the side/lateral view. (1) denotes the body shell of the mask (2) denotes the Adaptor end where a specialised adaptor is connected. The specialised adaptor (3) has one proximal port (4) and two distal ports [shell port (5) and Dynacuff port (6)J. The proximal port is connected to the ventilator circuit through a standard connector. The shell port of the adaptor connects to the respiratory gas inlet of the shell body. The Dynacuff port of the specialised adaptor connects to the Dynacuff port of mask which leads to the dynamic cuff through a dedicated channel (8)

The Mask may be made of rigid plastic or any suitable transparent or opaque material. The specialised adaptor is made of rigid plastic or any other suitable material. The dynamic cuff (10) and the static cuff (9) are made either with polythene/silicone/latex rubber or any other suitable material.

Figure No 2 shows the specialised adaptor. Its proximal end contains the ventilator circuit port which contains a standard 22 mm male/15 mm female connector which can be connected to ventilator circuits or AMBU/other similar resuscitator bag. The mask end of the dedicated adaptor has two ports of which the ‘shell port’ (5) gets connected to the air inlet port of the shell of the mask and the other port “Dynacuff port” (6) gets connected to the air inlet port (4) which leads to the dynacuff (10) through a dedicated channel (8).

Figure No. 3 is showing the device applied on the patients face and the showing the inflation of dynamic cuff during inspiratory phase.

Figure Number 4 is showing the device applied on the patients face and the showing the deflation of dynamic cuff during expiratory phase.

We claim:

1: this device a face mask which can be used in patients who are spontaneously breathing and also on patients who require positive pressure ventilation This device has a main body or shell which has adaptor end and the patient and

[symbol]

2: the face mask assembly as set forth in 1 wherein the adaptor end of the shell is connected to a specialised adaptor having three ports of which the largest “proximal port” is connected to a ventilator circuit or other ventilating Apparatus like the Ambu resuscitator

3: a specialised adaptor as set forth in claim 2, wherein, the smaller port at the distal end of the specialised adaptor is connected to a port which inflates the dynamic cuff through a dedicated channel.

4: a face mask assembly as set forth in claim 1, which has one or more dynamic cuffs, positioned above a conventional static cuff, winch when placed on the patient's face moulds and assumes the contours of the patient's face thereby producing an airtight seal.

5: a dynamic cuff as set forth in claim 4, which closely mimics, but exceeds the Airway pressure of the patient's Airway in different phases of respiration as reflected in the Shell of the face mask assembly, thereby transmitting the pressure on the static cuff which is in contact with the face the patient, thereby creating a pressure on the face which exceeds the airway pressure during positive pressure ventilation thereby ensuring an airtight seal.

6: dynamic cuff as set forth in claim number 4 which expands by flow of anaesthetic gases/air to the cuff through the dedicated adaptor port and channel

7: a dynamic cuff as set forth in claim number 4 which has one or more pores on the inner side (towards the shell) of the cuff which directly communicate with the intra shell portion of the face mask assembly at the end of respiratory cycle washing out the exhaled gases into the ventilator circuit thereby reducing the Dead Space of the Apparatus

8: a dynamic stuff as set forth in claim number 4, which has a foam like sponge made of polyurethane or similar material inside its lumen as an alternative embodiment thereby providing better airway seal and patient comfort

9: a face mask assembly as described in claim No 1, which has a static cuff at the patient end

10: static cuff as set forth in claim number 9 which has a dedicated inflation port

11: a static cuff of as set forth in claim number 9 which may have a sponge like foam inside its lumen as an alternative embodiment

12: the static cuff as set forth in claim No 9, has adhesive strips along its margin which comes into contact with the face of the patient

13: the Dynamic cuff of as set forth in claim No 4, wherein its position interchanged with the static cuff as in claim No 9. The interchange of the position the static and dynamic cuffs is an alternative embodiment

14: In alternative embodiments, the Static and Dynamic cuffs as set forth in claims 9 and 4 respectively can be placed one inside the other instead of one over the other position as set forth in the preferred embodiment: i.e, the static cuff can be placed inside the dynamic cuff surrounded by the latter on all directions and vice versa without deviating from the principle of this invention.

15: the Dynamic cuff as in the alternative embodiment set forth in claim 13 which has adhesive strips in the margin which is coming into contact with the face of the patient

16: the face mask assembly as set forth in claim No 1 which has a circular ring around the air inlet port of the cell which has 4 or more hooks

17: the circular ring h hooks as set forth in claim No 15 connected to harness belt made of stretchable material preferably silicone/Polythene/latex rubber or other similar materials enabling airtight approximation of the face mask against the face of the patient

18: the circular ring as set forth in claim No 15 which can be connected with a headgear enabling airtight positioning of the face mask assembly over the face of the patient

19: face mask assembly as set forth in claim No 1 which has inbuilt fragrant material making it pleasant for children
